(19) United States
(12) Patent Application Publication
(10) Pub. No.: US 2014/0031303 A1
(43) Pub. Date: Jan. 30, 2014

(54) Bioactive hybrid of ionic/polyhydroxyl, lipophilic and covalently reactive moieties and method of use

(71) Applicants: William Lewis, Chula Vista, CA (US); Terry Elaine Lewis, Chula Vista, CA (US)

(72) Inventors: William Lewis, Chula Vista, CA (US); Terry Elaine Lewis, Chula Vista, CA (US)

(21) Appl. No.: 13/741,281

(22) Filed: Jan. 14, 2013

Related U.S. Application Data
(60) Provisional application No. 61/588,879, filed on Jan. 20, 2012.

(51) Int. Cl. A61K 31/7028

(52) U.S. Cl. CPC A61K 31/7028
USPC 514/25

(57) ABSTRACT

A combination of the following three structures in a single molecule imparts a surprisingly broad range of beneficial effects in promoting and maintaining health. First, a strongly hydrophilic moiety, either a cation or anion or a polyhydroxyl group, with two or preferably three or more hydroxyl groups. Second, a lipophilic group, such as an alkyl chain of at least 8 carbons, and preferentially more, up to 18 or even 30-odd atoms in the chain. And third, a covalently reactive group, preferably with a strong tendency for binding to biosurfaces and biomolecules such as proteins, cell membranes, and/or nucleic acids, such as an epoxide, an acid anhydride, isocyanate or other reactive group(s) as herein defined.

### CROSS-REFERENCE TO RELATED APPLICATIONS

“This application claims the benefit of U.S. Provisional Application No. 61/588879, filed Jan. 20, 2012.”

### STATEMENT REGARDING FEDERALLY SPONSORED RESEARCH OR DEVELOPMENT

Not Applicable.

### BACKGROUND OF THE INVENTION

### Introduction

This invention relates to the field of health, particularly of homeostasis and healing. It relates to homeopathic practice and to nutritional supplements. The inventive compounds provide support to homeostasis by facilitating immune system stimulation, microbial resistance, pain-relief, and/or stimulation of healing or natural growth. They are especially active by direct application to the cells in the area to be treated, or by introduction as close as is practical.

### What's Been Done, with its Limits

Many beneficial compounds are known, for establishing and maintaining desirably good health, including foods, vitamins and other supplements, pharmaceuticals, homeopathic remedies, herbs and various preparations of other types. Each has its range of use, in terms of dose, indications for use, beneficial effects, and unwanted “side effects.” Even food can have an unwanted side effect of allergies, bloating or obesity, for example. Vitamins such as vitamin A, can be toxic in doses that exceed the desirable amount. And pharmaceuticals are known to always carry the risk of significant side effects that can be unpleasant or even life-threatening.

An additional problem that often occurs with current pharmaceuticals is that they are only effective on very narrow targets, such as a single enzyme, ion channel or receptor. So a particular medicine has only a very limited range of use. This multiplies the number of medicines required to maintain good health, and contributes to the cost of medications—both in the number required by a particular user, and the number that must be developed, proven out, and manufactured. Generally pharmaceuticals are increasingly complex molecules, further adding to their manufacturing cost.

Some classes of medications have serious negative effects on the immune system, and in general they do not work well in synergy with it. Steroids are avoided by many people because, although they may help temporarily with symptom reduction, they have widespread effects in depressing the body's natural defenses. So, for example, a friend with asthma recently died of lung cancer though he had never smoked, probably because the steroidal inhalant he used compromised his immune system in his lungs.

With pharmaceuticals mainly acting on very narrow targets, there is a huge problem of microbes developing resistance to them by small mutations in their structure. When resistance builds up, then it is often necessary to start research from scratch, to try to attack a different target in the invading microbe. And the ease of mutation of various microorganisms leads to a sense of impotence and fear in facing increasingly-resistant microbes that may one day cause pandemics with no known cure.

Because of the complexity of the molecules used as pharmaceuticals, their break-down products may not easily be metabolized, and may persist in the body, leading to the aforementioned side effects. That is, while the parent molecule used for a pharmaceutical benefit has one biological action that is good, the molecule may be broken down or metabolized into other “unnatural” structures that have negative effects, until they are eventually completely metabolized. Thus, in western medicine, it is often the case that the health practitioner is attempting to kill the invader slightly faster than he is killing the host—the patient.

### What's Needed (Advantages of the New Approach)

What has been needed is a new approach to creating bioactive molecules that are beneficial to health, and which the present invention provides. Ideally, the new therapeutic compounds would have a broad spectrum of action, so that a single formulation would have many uses. This would help reduce the shelf space and complexity related to providing and taking therapies, and reduce development and manufacturing costs, especially if the compounds were relatively simple.

Likewise, preferable new compounds would have more than one mode of action, to not only target broad classes of microbes, but also to activate the immune system for attacking invading microbes, and/or for replacing damaged tissues by healthy rebuilding. Working with the immune system, instead of apart from, or even against it, would be a great advantage. Another possible beneficial action that the new compounds could have, would be to reduce pain, and/or to reduce tissue swelling, blistering, or other discomfort.

Ideally, the compounds would affect foreign bodies (microbes, etc.) in a way that would reduce or eliminate their viability, and possibly “flag” them for attack and destruction by the immune system—especially those microbes which seem to have found ways of “hiding” in the body and avoiding being attacked by the body's defenses.

Of course, it would be preferable if the break-down/by-product/metabolite products of the therapeutic compounds were innocuous and readily converted or recycled into useful materials or easily eliminated without causing negative effects in the quantities used.

If it were difficult for microbes to mutate sufficiently to become immune to the effects of the new compounds, that could make the compounds useful during a greater number of years than pharmaceuticals now used. And if it were then easy to use different molecular structures to accomplish the same overall purpose, the new class of compounds could be useful for a very long time.

Instead of using the “western medicine” principle of killing the invader faster than killing the patient, it could be advantageous to use the homeopathic principle that “a little poison” can have a large positive health effect. That is, not that the compound should be dangerous or poisonous, but that it leverages its activity so that a small dose can be very effective.

Although not a requirement of an ideal new approach to improving health, it may be noted that the present inventors believe in the “germ theory” of disease, that microbes are in most cases—more often than is generally realized—the cause of poor health, that more and more diseases are likely to be found to be precipitated by the presence of foreign bodies—whether bacteria, viruses, prions or others—and that helping the immune system to seek out and destroy these invaders, is a powerful approach to improving health.

### Why the New Approach Works

We do not know exactly why this new class of compounds we present as our invention works. We have theories, and offer them as possibilities that may assist other researchers in expanding knowledge about this surprisingly effective class of chemicals.

We believe that the chemicals of this invention attack many sites by virtue of their chemical reactivity to moieties—chemical functional groups such as alcohol, acid, olefins, etc.—that are present in various biomolecules. The chemicals probably attack both microbes and body tissues, and thereby modify proteins, dna, saccharides and other biomolecules. The immune system “recognizes” the difference in the structure, as being “unnatural.” If an invader, it is killed, if possible. If body tissue, it is rebuilt.

However, some of the actions of the new compounds, such as pain relief, we do not yet have a clear theory about. Presumably there is some effect on the ion channels that mediate pain signaling and transmission. In any case, the validity and value of the invention rests on the effects of these compounds, not on theories about how they accomplish their beneficial actions.

As we accumulated familiarity with this new beneficial class of compounds, the following attributes became important, for meeting the goals of an improved and preferred approach to health benefits. The molecules apparently needed to have a certain solubility in bodily fluids and tissues, a definitely-reactive moiety, and an ability to be metabolized to innocuous (perhaps even beneficial) products.

This invention comprises tri-functional molecules that provide a surprising range of beneficial effects, but have conversely shown a surprising lack of side effects. That is, the therapeutic ratio of these compounds is high.

### BRIEF SUMMARY OF THE INVENTION

We have found that the combination of the following three structures in a single molecule imparts a surprisingly broad range of beneficial effects in promoting and maintaining health. First, a strongly hydrophilic moiety, either a cation or anion or a polyhydroxyl group, with two or preferably three or more hydroxyl groups. Second, a lipophilic group, such as an alkyl chain of at least 8 carbons, and preferentially more, up to 18 or even 30-odd atoms in the chain. And third, a covalently reactive group, preferably with a strong tendency for binding to biosurfaces and biomolecules such as proteins, cell membranes, and/or nucleic acids, such as an epoxide, an acid anhydride, isocyanate or other reactive group(s) as herein defined.

### DETAILED DESCRIPTION OF THE INVENTION

We do not have a comprehensive understanding of the mechanisms of action of these molecules. In some cases, it appears that the molecule may act by inserting into and disrupting membranes. In some cases, the binding of the molecule to foreign bodies such as microbes, seems to provide an alteration of surface structure of the microbe, such that the host's immune system more readily recognizes it as exogenous and is more strongly activated to respond to and attack/eliminate the “invader.” We also believe that, by binding to some endogenous molecules, that some of the host's own cell components may also be attacked by the immune system, but that the general activation of the immune system that follows has a beneficial effect in healing and restoration of homeostasis. We believe that when endogenous molecules are modified by attachment of the molecules of the invention, that the body probably can recognize and remove the attached moiety without needing to totally break down the molecule or cell that has been modified. But when a foreign body is modified, the body recognizes as it breaks it down, that it is not “normal” or endogenous material, and continues to break it down until the foreign body is eliminated.

Another possible mode of action of these molecules may be that they alter the permeability or membranes, or the uptake or release of molecules or ions into or out of cells. This may be a factor in the perceived pain relief often experienced. It is also possible that they provide important cellular components in a bio-available form. This may be important in some cases, such as in the re-initiation of hair growth.

The polyhydroxyl group is necessary to the molecule to provide a moderate degree of hydrophilicity. This helps with solubility in tissues, and may help with disruption of cell membranes. An example of a polyhydroxyl group is a saccharide moiety. At least two hydroxyl groups must be present, but need not be on adjacent members of a chain or ring.

The hydrophobic group is necessary to the molecule to provide hydrophobicity, and we believe it may also be important that it have the ability to insert into cell membranes or other distinct cellular structures. The most preferred of these moieties are those that are actually present in various cell membranes, such as a palmityl, linolenyl, or oleyl group, or others described below. However, “unnatural” groups may also be used, as long as they have similar hydrophobicity and preferentially, the ability to insert into biological membranes and/or cellular structures. The hydrophobic chain is not required to consist only of carbon and hydrogen atoms. Heteroatoms such as halides, oxygen, sulfur, etc. may be tolerated and even beneficially modify the overall properties of the molecule. The degree of hydrophobicity needed at a minimum is that provided by an n-octyl group. This may be measured by testing the water solubility of the corresponding alcohol in comparison to n-octanol. In the case of complex structures, the hydrophobicity may be calculated by known principles, or be inferred by comparison tests vs. a comparable molecule having an n-octyl group providing hydrophobicity. The hydrophobic group may consist of a single chain or multiple substituents on the compound of the invention, including aliphatic, aromatic and heterocyclic ring(s).

Naturally occurring lipids provide an especially useful starting point for preparation of compounds of the invention, both because they are renewable resources, and because they are likely to provide effective (that is, biologically active) interactions with living cells as part of a compound of the invention. Here are commonly known lipids:

Fatty acids: Lauric (12 carbons), palmitic (16 carbons), stearic (18 carbons), lignoceric (24 carbons), oleic (C-18, one double bond), nervonic (C-24, one double bond), arachidonic (C-20, 4 double bonds), and eicosapentaenoic (C-20, five double bonds) are examples of fatty acids, that is, carboxylic acids with hydrophobic tails.

Fatty alcohol examples: Melissyl (C-30) alcohol is a long-chain fatty alcohol present in beeswax as the palmityl ester.

Triacylglycerols (fats and oils derived from plants or animals), and/or diacylglycerols (present in small amounts in natural fats and oils, and at higher levels in Enova brand enzyme-processed oil) or monoacylglycerols may provide hydrophobic groups for preparation of compounds of the invention. They are glycerol (poly)esters of various fatty acids.

Naturally-occuring glycerophospholipids are diacylglycerols further esterified to phosphoric acid, which may have ethanolamine, choline, serine, or inositol also esterified with the acid. Those with inositol thus contain both lipophilicity and a polyhydroxyl group. Lecithin is a phosphatidylcholine (that is, a phosphoric acid diester of diacylglycerol and choline) and is found particularly in nervous tissue such as the white matter of brain, nerves, neural tissue, and in spinal cord. Phosphatidylethanolamines, also called cephalins, are the principal phospholipids in bacteria, whereas lecithins are the principal phospholipids in animals.

Sphingophospholipids comprise the other major group of natural phospholipids, and are also found in cell membranes. Sphingosine is a dihydroxyamine (specifically, 1-hydroxymethyl-3-hydroxyheptadec-3-enylamine) that in sphingomyelins is derivatized to the amide with a fatty acid (C-18 to C-24) esterified (at the 1-hydroxymethyl group) along with choline to phosphoric acid). Thus, it has some structural analogy to phosphatidyl cholines, but has the lipophilic sphingosine-amide instead of a glycerol diacyl ester. It is abundant in brain and nerve tissue, as coating around nerve fibers (the myelin sheath).

Sphingoglycolipids (cerebrosides) are lipids composed of a sugar (the “glyco”moiety) connected by an acetal or ketal linkage to the 1-hydroxymethyl group of the amino alcohol sphingosine, which also has an unsaturated fatty acid (as the amide). The sugar is usually a monosaccharide such as glucose or galactose. Cerebrosides are abundant in brain tissue.

Phosphoglycerides comprise the major lipid component of cell membranes, along with spingomyelin and cholesterol.

Steroids are lipids with a specific arrangement of four fused hydrocarbon rings, and many have OH groups that provide potential linkage points for preparation of compounds of the invention. They are biosynthesized from the pyrophosphate ester of farnesol (a 15-carbon acid with 4 methyl groups and 3 non-congugated double bonds) via squalene (a dimer of the farnesyl group), both of which compounds may serve as precursors to compounds of the invention.

Cholesterol is the most abundant steroid in the human body, and an essential component of cell membranes. Adrenocortical steroids (secreted by the adrenal glands near the upper end of each kidney) include cortisol and aldosterone. Sex hormones include testosterone, estradiol, and progesterone (ketone group; no OH group). Mineralocorticoids (which control tissue swelling by regulating cellular salt balance) include aldosterone. Glucocorticoids such as hydrocortisone, are involved in regulation of glucose metabolism and control of inflammation.

Bile salts, including sodium glycocholate and sodium taurocholate, help in the small intestines as soaps to break up fats into micelles for more effective digestion.

Eicosanoids include leukotrienes (involved in inflammation and asthma), prostaglandins, and thromboxanes (vasoconstrictors and facilitators of platelet aggregation).

Prostaglandins are C-20 lipids (fatty acids) that contain a five-membered ring with two long side chains, and have effects ranging from lowering blood pressure, lowering gastric secretions & protecting the stomach, and controlling kidney function & contractions in birth. They can also cause constriction or dilation in vascular smooth muscle cells, cause aggregation or disaggregation of platelets, sensitize spinal neurons to pain, decrease intraocular pressure, regulate inflammatory mediation, regulate calcium movement, control hormone regulation, and control cell growth. They are biosynthesized from linoleic acid via arachidonic acid (C-20 unsaturated fatty acid). They are key in sending out pain and inflammation signals to the body. NSAIDS and other pain relievers appear to function by blocking the COX enzymes that produce prostaglandins.

The covalently reactive group may be any of such as are known in the art. It may be any group with a strong tendency to bind to the organic molecules of cells and/or pathogens. It may be a reactive species such as an acid chloride or anhydride. The acid may be a carboxylic acid, or other acid such as sulfonic, sulfinic, sulfuric, phospinic, phosphonic, phosphoric, etc.

The covalently reactive group may provide reactive additions to carbon-carbon double bonds, such as hydroboration and hydrosilylation and free radical precursors. These latter may also/alternatively provide grafting by substitution on molecules in or of the treated organism. The covalently reactive group may provide substitution at carbon-halogen moieties , including using thiols. Epoxy groups may be used as the binding group. Sulfides and sulfoxides, phosphine and phosphite groups can react with alkyl chloride groups in the organism. Phosphorus or sulfur ylides may react with aldehyde or ketone groups. Other reactive groups or other reactive species such as are known or may become known in the art may also be used.

The covalently reactive group must be capable of forming a (polar or non-polar) covalent bond with molecules in living cells at ambient temperature, not merely ionic or hydrogen bond or non-polar attractions. The reactivity must be sufficient to yield at least 10% reaction in two hours at room temperature in water or physiological fluid at a pH encountered in bodily fluids, cells or organelles within cells. In addition to groups meeting this standard, groups comprising a silicon, germanium or boron atom connected to one or more hydroxyl groups are included as “covalently reactive groups.”

At least one covalently-reactive/binding-tendency group must be present, but additional such groups may be present and may or may not provide beneficial effects. Likewise, there may be more than one lipophilic and/or more than one polyhydroxyl group in the molecule.

The distance between the binding group and the polyhydroxyl moiety or moieties may vary and the linking chain may be polar, non-polar, substituted or unsubstituted, straight, branched or cyclic. It may comprise an all-carbon backbone, or contain other linkages such as ether, ester, amide, etc.

The inventive compounds may be used alone, or with appropriate solvents, carriers, adjuvants, stabilizers, synergists, etc., as are known, or may become known in the art.

### Definitions

Benefit=an improvement in the health or natural functioning of the cell or organism, relative to the state present at the time of treatment, or that would be or become present if treatment were withheld. This includes, inter alia, promotion of healing, replacement of diseased tissue with healthy growth, initiation of growth that had ceased such as of hair, prevention of damage due to external factors such as heat, insolation or other radiation, and/or prevention or rejection of infection.

Living cells and organisms=any living cell, whether a single-cell organism, or multi-cell organisms such as microbes, plants, and animals Includes spores, seeds, etc., but not viruses or prions.

Introduction=act or function of bringing a compound of the invention into contact with the cell(s) to be benefited, whether by any method of administration, including but not limited to infusion, perfusion, topical application (e.g., epicutaneous, inhalational, via enema, or by application to the eye, ear, nose or vagina), enteral (e.g., orally, by feeding tube, or by suppository), parenteral by injection or infusion/perfusion (e.g., intravenous, intraarterial, intramuscular, intracardiac, subcutaneous, intraosseous infusion, intradermal, intrathecal, intraperitoneal or intravesical), other parenteral (e.g., inhalational, transdermal or transmucosal including insufflation, sublingual or buccal), including slow or immediate release, or use of carrier, clathrate, etc.

High therapeutic ratio=in a representative population of the targeted species, 90% or more of individuals treated receive more benefit than harm from an optimized dosage in targeting a desirable effect.

Not naturally present=the trifunctional compound used, is not naturally produced by the cell or organism treated.

### EXAMPLES

The following examples illustrate the invention, but do not limit its scope:

Examples of the inventive compounds are compounds, EP-23, JX-153, CHC-36, AM-59 and TA-21, obtained under a research agreement from Abchem Manufacturing Co., Chula Vista Calif. 91911. We have found them to be beneficial in the treatment of many abnormalities in health of humans, animals and plants. Each comprises a reactive salt with a lipophilic tail, within the parameters specified above.

### Example 1

Pain due to thermal or sun or radiation therapy burns, shingles, acid reflux, pimples and insect bites, was strongly reduced by application of a dilute water or alcohol solution of EP-23 to the body. In the case of the insect bites, there was significant itch reduction. Typically, pain and itch relief lasted 3 or 4 hours, and was renewed by a subsequent application.

### Example 2

Wounds, such as cuts, scrapes, burns, and persistent sores healed rapidly and without infection after treatment as in Example 1. Typically treatment was once daily, or more often if pain relief was desired.

### Example 3

Relief from various ailments normally thought to be due to viral action, such as cold sores, plantars warts, canker sores, genital herpes, warts on vocal cords, molluscum contagiosum, and stomach ailments was found to reliably follow treatment with JX-153 as in example 2. Pain relief was also observed.

### Example 4

Pre-cancerous and cancerous lesions that had persisted on the skin were treated with CHC-36 as in Example 2, or less often. The pre-cancerous spots were apparently healed from underneath, with the tissue on top eventually falling off. The lesions healed promptly.

### Example 5

Warts and moles were eliminated by treatment with JX-153 as in Example 3. The warts appeared to be extruded from the skin by growth from underneath, over a period of weeks. The moles gradually fell off, similar to the pre-cancerous spots. However, the moles most readily removed were “raised” areas on the skin, whereas flat “discrepancies” on the skin did not respond significantly to the JX-153. Skin tags fell off, replaced with normal skin, but only after prolonged treatment of more than a month.

### Example 6

Abnormalities of the skin thought to be due to fungal patches, where the color of the skin was bleached vs. normal, prior pigmentation, were returned to normal by treatment as in Example 4. Likewise, skin abnormalities under or adjacent to finger and toe nails, that were thought to be due to fungus, were improved by treatment for weeks or months.

### Example 7

Abnormalities thought to be due to bacterial action, such as against cystic and regular acne, earache, pinkeye, and traveler's diarrhea were resolved after treatment with AM-59 as in Example 4. Interestingly, earache was permanently relieved by only a few applications on the external ear—the expanded portion named the auricula or pinna—rather than requiring instillation into the external acoustic meatus, i.e., the ear canal, which would presumably be much closer to the infection.

### Example 8

Stimulation of tissue growth was observed in cases such as re-growing the severed tip of a finger and in healing/re-growing of gum after surgery and/or dental disease, by treatment using a solution of TA-21 as in Example 4—in the latter cases, as a mouth rinse.

### Example 9

Stimulation of bone growth, such as re-attachment of loose teeth to the jaw, was observed by periodic rinsing of the mouth with a solution of TA-21 as in Example 4.

### Example 10

Stimulation of tooth enamel growth for healing of dental caries was accomplished with treatment with TA-21 as in Example 4, so that a cavity of about 0.3×1 mm was replaced by normal tooth and enamel.

### Example 1

Treatment of bald areas of scalp with TA-21 as in Example 4 has stimulated hair growth, for reversal of balding, in areas where there had been no hair for many years. The hair began growing within a few weeks, as “baby hair,” with gradual maturing with continued growth.

### Example 12

Blistering after abrasion or burning was significantly reduced by even a single timely application of EP-23 solution as in Example 1.

### Example 13

A strong reduction in pain, likened to the effectiveness of the strong painkiller “Demerol,” was obtained, along with a strong reduction in the burn-like effects on the skin, by treatment of areas of skin that were subjected to radiation therapy against cancers of the breast in several cases, and of an arm in another case, using a solution of EP-23 as in Example 4.

We claim:

1. A method of providing a benefit or benefits to living cells and organisms comprising the introduction of an effective amount of one or more high-therapeutic-ratio trifunctional compounds to them, that are not naturally present, wherein the three functional groups comprise (a) a polyhydroxyl or ionic group or groups, (b) a hydrophobic moiety or moieties of aggregate hydrophobicity at least equal to that of an n-octyl group, and (c) one or more covalently reactive groups.

2. A composition of matter comprising a high-therapeutic-ratio trifunctional compound, wherein the three functional groups comprise (a) a polyhydroxyl or ionic group or groups, (b) a hydrophobic moiety or moieties of aggregate hydrophobicity at least equal to that of an n-octyl group, and (c) one or more covalently reactive groups.

3. A method of claim 1, wherein the trifunctional compound is EP-23, JX-153, CHC-36, AM-59 or TA-21.
